(19) United States
(12) Patent Application Publication
(10) Pub. No.: US 2026/0153521 A1
(43) Pub. Date: Jun. 4, 2026

(54) DIAGNOSTIC SYSTEM FOR HEMOGLOBIN ANALYSIS

(71) Applicant: CASE WESTERN RESERVE UNIVERSITY, Cleveland, OH (US)

(72) Inventors: Umut Gurkan, Cleveland, OH (US); Ran An, Shaker Heights, OH (US); Zoe Sekyonda, Cleveland, OH (US); Arwa Fraiwan, Cleveland, OH (US)

(21) Appl. No.: 18/706,579

(22) PCT Filed: Nov. 1, 2022
(86) PCT No.: PCT/US2022/079062

§ 371 Date: May 1, 2024

Related U.S. Application Data
(60) Provisional application No. 63/274,299, filed on Nov. 1, 2021.

(51) Int. Cl. G01N 33/72; B01L 3/00; G01N 27/447

(52) U.S. Cl. CPC G01N 33/726; B01L 3/502715; B01L 3/50273; G01N 27/44704; B01L 2200/16; B01L 2300/047; B01L 2300/0645; B01L 2300/0663; B01L 2300/0825; B01L 2300/12; B01L 2400/0421; G01N 2800/042; G01N 2800/50

(57) ABSTRACT

Embodiments described herein relate to a diagnostic system and electrophoresis device for detecting and/or measuring hemoglobin variants in blood of a subject. The diagnostic system includes a cartridge and a UV imaging system. The cartridge includes a housing having a microchannel extending between first and second buffer pools each containing a buffer solution. The buffer solution exhibits an affinity to non-glycosylated hemoglobin, which facilitates its separation from glycosylated hemoglobin. An electrophoresis strip is positioned within the microchannel and is structured to receive at least a portion of a hemolysate. The electrophoresis strip has first and second ends positioned in the first and second buffer pools so as to be at least partially saturated with the buffer solution in each buffer pool, with a first electrode exposed to the buffer solution in the first buffer pool, and a second electrode exposed to the buffer solution in the second buffer pool.

### RELATED APPLICATION

This application claims priority from U.S. Provisional Application No. 63/274,299, filed Nov. 1, 2021, the subject matter of which are incorporated herein by reference in their entirety.

### FIELD OF THE INVENTION

The present invention is related to a diagnostic system, and particularly relates to a diagnostic system that includes an electrophoresis device that rapidly and easily perform blood analysis.

### BACKGROUND

Monitoring of the course of diabetes in a patient may be accomplished by checking the glucose level in the blood. However, changes in this level are known to be especially rapid. Glucose assays can give only sporadic information about the patient's blood sugar level, and hence do not reflect the changes in the latter in the weeks preceding the analysis.

Quantitative determination of glycosylated hemoglobin A1 (HbA1c) is known to reflect a patient's average blood glucose concentration over a period of two months preceding the taking of a blood sample. HbA1c is defined by the International Federation of Clinical Chemistry working group (IFCC) as hemoglobin that is irreversibly glycated at one or both N-terminal valines of the beta chains. It is formed from irreversible, slow, non-enzymatic addition of a sugar residue to the hemoglobin, and the rate of production is directly proportional to the ambient glucose concentration. The long lifespan of erythrocytes (mean 120 days) enables HbA1c to be used as an index of glycemic control over the preceding two to three months and as the adequacy of treatment in diabetic patients. For this reason, HbA1c is widely used in a screening test for diabetes mellitus and as a test item for checking whether a diabetic keeps the blood sugar under control.

Conventionally, HbA1c has been measured by HPLC, immunoassay, electrophoresis or the like. HPLC is widely used in clinical examinations. HPLC requires only 1 to 2 minutes to measure each sample, and has achieved a measurement accuracy of about 1.0% in terms of a CV value obtained by a within-run reproducibility test. Measurement methods for checking whether a diabetic keeps the blood sugar under control are required to perform at this level.

Measurement of hemoglobin by electrophoresis has been used for a long time to separate abnormal Hbs with an unusual amino acid sequence. However, separation of HbA1c is significantly difficult, and takes 30 minutes or more by gel electrophoresis. Thus, electrophoresis has been unsatisfactory in terms of measurement time and measurement accuracy when applied to the clinical examinations. Therefore, electrophoresis has hardly been applied to clinical diagnosis of diabetes.

### SUMMARY

Embodiments described herein relate to a diagnostic system and electrophoresis device for detecting and/or measuring hemoglobin variants in blood of a subject, and particularly relates to a cartridge of an electrophoresis device for a point-of-care diagnostic system for measuring hemoglobin (Hb) types, such as HbA1c, in a subject to determine blood glucose concentration. In some embodiments, the diagnostic system can be used to measure HbA1c levels to determine glucose levels in a subject having or suspected of having diabetes.

In some embodiments, the diagnostic system includes a cartridge and a UV imaging system. The cartridge includes a housing having a microchannel extending between first and second buffer pools each containing a buffer solution. The buffer solution exhibits an affinity to non-glycosylated hemoglobin, which facilitates its separation from glycosylated hemoglobin. An electrophoresis strip is positioned within the microchannel and is structured to receive at least a portion of a hemolysate. The electrophoresis strip has first and second ends positioned in the first and second buffer pools so as to be at least partially saturated with the buffer solution in each buffer pool. A first electrode is connected to the housing and is exposed to the buffer solution in the first buffer pool, and a second electrode is connected to the housing and is exposed to the buffer solution in the second buffer pool. The first and second electrodes are configured to generate an electric field across the electrophoresis strip. The application of the electric field to the first and second electrodes induces migration and separation of bands of non-glycosylated hemoglobin and glycosylated hemoglobin in the hemolysate delivered to the electrophoresis strip. A portion of the housing is optically transparent for visualizing the migrated and separated bands of non-glycosylated hemoglobin and glycosylated hemoglobin on the electrophoresis strip. The UV imaging system is configured to illuminate the bands of non-glycosylated hemoglobin and glycosylated hemoglobin with UV radiation, detect through the optically transparent portion of the housing the bands of non-glycosylated hemoglobin and glycosylated hemoglobin on the electrophoresis strip caused by the applied electric field, and generate band detection data based on the bands of non-glycosylated hemoglobin and glycosylated hemoglobin.

In some embodiments, the UV imaging system includes a UV source that illuminates the bands with UV radiation at a wavelength of about 400 nm to about 420 nm.

In some embodiments, the diagnostic system includes a processor configured to receive and analyze the band detection data to determine one or more band characteristics for the bands and generate diagnostic results based on the one or more band characteristics.

In some embodiments, the UV imaging system is configured to detect at least one of band size, migration, and intensity, and the processor includes a decision algorithm configured to generate density plots based on the band size, migration, and intensity and identify the relative percentages of glycosylated and non-glycosylated hemoglobin bands based on the density plots. The processor can also be configured to diagnose whether the subject has or is at risk of diabetes based on the relative percentages of the glycosylated and non-glycosylated hemoglobin bands.

In some embodiments, the buffer solution is acidic and includes a sulfated polysaccharide.

In some embodiments, the system further includes a power supply connected to the first electrode and the second electrode that is configured to supply the electric field across the electrophoresis strip.

In some embodiments, the system includes a buffer replenishing system configured to replenish the buffer solution for the first and second buffer pools and counteract pH changes in the buffer solution during operation of the system. The buffer replenishing system can include a first inlet conduit and a first outlet conduit in fluid communication with the first buffer pool and a second inlet conduit and a second outlet conduit in fluid communication with the second buffer pool for supplying and removing buffer solution from, respectively, the first buffer pool and the second buffer pool.

In some embodiments, the buffer solution can flow at a rate of about 3 μl/min to about 8 μl/min through the first inlet conduit and the second inlet conduit to the first buffer pool and the second buffer pool and at a rate of about 1 μl/min to about 3 μl/min from the first buffer pool and the second buffer pool through the first outlet conduit and the second outlet conduit.

In some embodiments, the first electrode and the second electrode are at least partially embedded in and extend, respectively, substantially orthogonal to a length of the electrophoresis strip in the first buffer pool and second buffer pool.

Other embodiments described herein relate to a diagnostic system for identification and quantification of glycosylated hemoglobin and non-glycosylated hemoglobin in a blood sample. The diagnostic system includes a cartridge, a UV imaging system, and a processor. The cartridge includes a housing having a microchannel extending between first and second buffer pools each containing a buffer solution. The buffer solution exhibits an affinity to non-glycosylated hemoglobin, which facilitates its separation from glycosylated hemoglobin. An electrophoresis strip is positioned within the microchannel and structured to receive at least a portion of a hemolysate. The electrophoresis strip has first and second ends positioned in the first and second buffer pools so as to be at least partially saturated with the buffer solution in each buffer pool. A first electrode is connected to the housing and exposed to the buffer solution in the first buffer pool, and a second electrode is connected to the housing and exposed to the buffer solution in the second buffer pool. The first and second electrodes are configured to generate an electric field across the electrophoresis strip. The application of the electric field to the first and second electrodes induces migration and separation of bands of non-glycosylated hemoglobin and glycosylated hemoglobin in the hemolysate delivered to the electrophoresis strip. A portion of the housing is optically transparent for visualizing the migrated and separated bands of non-glycosylated hemoglobin and glycosylated hemoglobin on the electrophoresis strip. The UV imaging system is configured to illuminate the bands of non-glycosylated hemoglobin and glycosylated hemoglobin with UV radiation, detect through the optically transparent portion of the housing the bands of non-glycosylated hemoglobin and glycosylated hemoglobin on the electrophoresis strip caused by the applied electric field, and generate band detection data based on the bands of non-glycosylated hemoglobin and glycosylated hemoglobin. The processor is configured to receive and analyze the band detection data to determine one or more band characteristics for the bands and generate diagnostic results based on the one or more band characteristics.

In some embodiments, the UV imaging system is configured to detect at least one of band size, migration, and intensity and the processor includes a decision algorithm configured to generate density plots based on the band size, migration, and intensity and identify the relative percentages of glycosylated and non-glycosylated hemoglobin bands based on the generated density plots. For example, the processor can be configured to diagnose whether the subject has or is at risk of diabetes based on the relative percentages of glycosylated and non-glycosylated hemoglobin bands.

In some embodiments, the buffer solution is acidic and includes a sulfated polysaccharide.

In some embodiments, the system includes a power supply connected to the first electrode and the second electrode configured to supply the electric field across the electrophoresis strip.

In other embodiments, the system further includes a buffer replenishing system configured to replenish the buffer solution for the first and second buffer pools and counteracts pH changes in the buffer solution during operation of the system.

The buffer replenishing system can include a first inlet conduit and a first outlet conduit in fluid communication with the first buffer pool and a second inlet conduit and a second outlet conduit in fluid communication with the second buffer pool for supplying and removing buffer solution from, respectively, the first buffer pool and the second buffer pool.

In some embodiments, the buffer solution can flow at a rate of about 3 μl/min to about 8 μl/min through the first inlet conduit and the second inlet conduit to the first buffer pool and the second buffer pool and at a rate of about 1 μl/min to about 3 μl/min from the first buffer pool and the second buffer pool through the first outlet conduit and the second outlet conduit.

In some embodiments, the first electrode and the second electrode are at least partially embedded in and extend, respectively, substantially orthogonal to a length of the electrophoresis strip in the first buffer pool and second buffer pool.

### BRIEF DESCRIPTION OF THE FIGURES

FIGS. 1(A-B) illustrate (A) schematic view of diagnostic system in accordance with an embodiment described herein and (B) an exploded view of an example of a cartridge electrophoresis device.

FIG. 2 is a bottom view of the device of FIG. 1.

FIG. 3 is a front view of an indicating member of the device of FIG. 1.

FIG. 4 is a side view of the device of FIG. 1 and an enlarged portion thereof.

FIG. 5 is a section view of the device of FIG. 2 taken along line 5-5.

FIG. 6 is another side view of the device of FIG. 1 and an enlarged portion thereof.

FIG. 7 is a schematic illustration of the device of FIG. 1 in use.

FIGS. 8(A-E) illustrate another example cartridge electrophoresis device.

FIGS. 9(A-B) illustrate images showing time lapse photos and schematic illustrations of a electrophoresis test.

FIG. 10 illustrates a schematic showing a method of using the diagnostic system described herein.

FIGS. 11(A-D) illustrate HemeChip-GHb Point-of-Care (POC) for glycosylated hemoglobin (HbA1) and non-glycosylated hemoglobin (HbA) detection and quantification. (A) Exploded view of the HemeChip-GHb cartridge fabricated using lamination method from PMMA. Complete cartridge assembly consist of (top to bottom, (i) Plastic top, (ii) cellulose acetate paper, (iii) blotting pads, (iv) plastic middle part, (v) thin electrodes and (vii) bottom part with connected tubing) (B, C) HemeChip-GHb works on the principles of affinity cellulose acetate electrophoresis. (D) HbA1 and HbA are separated from a stamped blood sample, forming distinct bands on the cellulose acetate strip, based on their affinity in a precisely controlled electrical field in HemeChip-GHb followed by the separation of different individual HbA and HbA1.

FIGS. 12(A-C) illustrate glycosylated hemoglobin separation in HemeChip-GHb. (A): At t=0 the sample is at the loading without electric field. (B): Start of HbA1 electrophoretically separation from HbA within 10 minutes under electric field. (C): HbA1 Hemoglobin further separate from HbA by 20 mins under electric field.

FIG. 13(A-B) illustrate HemeChip-GHb quantification using 410 nm UV imaging (A) Healthy sample UV Image showing band migration generated electrophoretically and the density plot formulated from the images. HbA1 and HbA results (7.25% and 92.75 respectively) (B): Diabetic sample generated electrophoretically HbA1 and HbA results (22.6% and 77.4 respectively).

FIG. 14 illustrates association between HbA1 HemeChip-GHb and clinical reference standard HPLC values. Results demonstrate a close agreement between HemeChip-GHb measured HbA1 values and HPLC results, using the one-way Mann Whitney statistical analysis; Healthy samples (p=0.40, n=7) and diabetic (p=0.46, n=5) indicating a high accuracy of HbA1 measurement.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “microchannels” as used herein refer to pathways through a medium (e.g., silicon) that allow for movement of liquids and gasses. Microchannels thus can connect other components, i.e., keep components “in liquid communication.” While it is not intended that the present invention be limited by precise dimensions of the channels, illustrative ranges for channels are as follows: the channels can be between 0.35 and 100 μm in depth (preferably 50 μm) and between 50 and 1000 μm in width (preferably 400 μm). Channel length can be between 4 mm and 100 mm, or about 27 mm. An “electrophoresis channel” is a channel substantially filled with a material (e.g., cellulose acetate paper) that aids in the differential migration of biological substances (e.g., whole cells, proteins, lipids, nucleic acids). In particular, an electrophoresis channel may aid in the differential migration of blood cells based upon mutations in their respective hemoglobin content.

The term “microfabricated”, “micromachined” and/or “micromanufactured” as used herein, means to build, construct, assemble or create a device on a small scale (e.g., where components have micron size dimensions) or microscale. In one embodiment, electrophoresis devices are microfabricated (“microfabricated electrophoresis device”) in about the millimeter to centimeter size range.

The term “polymer” refers to a substance formed from two or more molecules of the same substance. Examples of a polymer are gels, crosslinked gels and polyacrylamide gels. Polymers may also be linear polymers. In a linear polymer the molecules align predominately in chains parallel or nearly parallel to each other. In a non-linear polymer the parallel alignment of molecules is not required.

The term “electrode” as used herein, refers to an electric conductor through which an electric current enters or leaves.

The term “channel spacer” as used herein, refers to a solid substrate capable of supporting lithographic etching. A channel spacer may comprise one, or more, microchannels and is sealed from the outside environment using dual adhesive films between a top cap and a bottom cap, respectively.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

The term “at risk of” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “derived from” as used herein, refers to the source of a compound or sample. In one respect, a compound or sample may be derived from an organism or particular species.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

Embodiments described herein relate to a diagnostic system and electrophoresis device for detecting and/or measuring hemoglobin variants in blood of a subject, and particularly relates to a cartridge for a point-of-care diagnostic system that includes a cartridge electrophoresis device for measuring hemoglobin (Hb) types, such as HbA1c. In some embodiments, the diagnostic system can be used to measure HbA1c levels to determine glucose levels in a subject having or suspected of having diabetes.

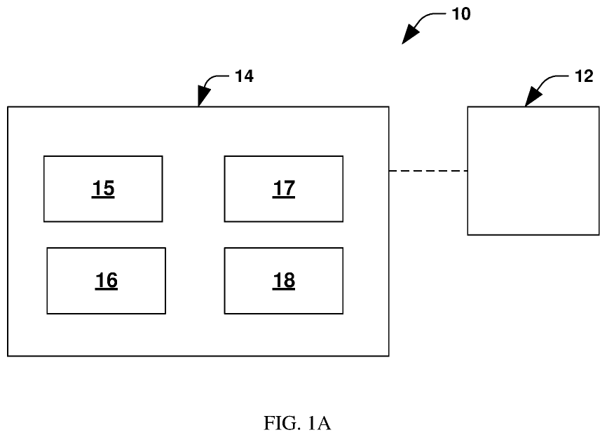

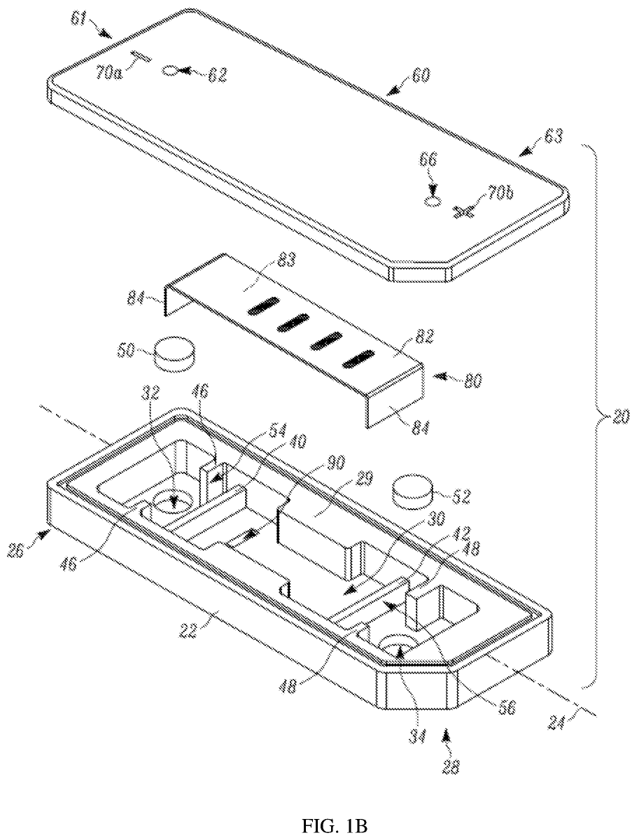

FIG. 1A is a schematic illustration of a point-of-care blood diagnostic system 10 in accordance an embodiment described herein. A point-of-care diagnostic system includes devices that are physically located at the site at which patients are tested and sometimes treated to provide quick results and highly effective treatment. Point-of-care devices can provide information and help in diagnosing patient disorders while the patient is present with potentially immediate referral and/or treatment. Unlike gold standard laboratory-based blood testing for disorders, the disclosed point-of-care devices enable diagnosis close to the patient while maintaining high sensitivity and accuracy aiding efficient and effective early treatment of the disorder and/or infection.

The diagnostic system 10 includes a cartridge electrophoresis device 12 for performing electrophoresis analysis on a sample and a reader 14 including a UV imaging system that can interface with the cartridge 14 to perform electrophoresis, analyze the electrophoresis, and optionally convey and/or display the result to a user of the system 10.

Referring to FIGS. 1B-3, an example cartridge electrophoresis device 20 includes a housing 22, an indicating member 80, and a cover 60. The housing 22 has a generally rectangular shape and extends along a centerline 24 from a first end 26 to a second end 28. A wall 29 of the housing 22 defines a recessed channel 30, i.e., microchannel, which extends between the first and second ends 26, 28. The microchannel 30 can be between about 0.35 and 100 μm in depth (preferably 50 μm) and between about 50 and 1000 μm in width (preferably 400 μm). The microchannel 30 length along the centerline 24 can be between 4 mm and 100 mm (preferably 27 mm).

The microchannel 30 is constructed to receive an electrophoresis strip sieving medium that aids in the differential migration of biological substances, such as whole cells, proteins, lipids, and nucleic acids. More specifically, the electrophoresis strip in the microchannel 30 is configured to suppress convective mixing of the fluid phase through which electrophoresis takes place and contributes to molecular sieving. In one example, the electrophoresis strip can constitute cellulose acetate paper. The electrophoresis channel 30 can aid in the differential migration of hemoglobin variants or types from a hemolysate of blood of a subject.

The wall 29 also helps to define first and second buffer pools 32, 34 located at opposite ends of the channel 30. More specifically, the first buffer pool 32 is positioned at or adjacent to the first end 26 of the housing 22. The second buffer pool 34 is positioned at or adjacent to the second end 28 of the housing.

A first opening 36 extends through the bottom of the housing 22 into the first buffer pool 32. A second opening 38 extends through the bottom of the housing 22 into the second buffer pool 34. As shown, the openings 36, 38 are circular. Alternatively, the openings 36, 38 can have any round or polygonal shape. In any case, an electrode 50 is positioned in the first opening 36 and exposed to the first buffer pool 32. An electrode 52 is positioned in the second opening 38 and exposed to the second buffer pool 34. The electrodes 50, 52 can be made from a conductive material, e.g., platinum, steel, 300 stainless steel, graphite and/or carbon.

In some embodiments, the first electrode 52 and the second electrode 54 can extend, respectively, substantially orthogonal to a length of the electrophoresis strip in the first buffer pool and second buffer pool.

A wall 40 is positioned within the microchannel 30 and helps define the boundary of the first buffer pool 32. The wall 40 spans the entire width of the microchannel 30 perpendicular to the centerline 24. A pair of restricting members 46 extends from the wall 29 of the housing 22 towards the centerline 24. The restricting members 46 extend parallel to the wall 40 and are positioned closer to the first end 26 than the wall. The restricting members 46 are spaced from one another and spaced from the centerline 24. A gap or space 54 is formed between the restricting members 46 and the wall 40.

A wall 42 is positioned within the microchannel 30 and helps define the boundary of the second buffer pool 34. The wall 42 spans the entire width of the microchannel 30 perpendicular to the centerline 24. A pair of restricting members 48 extends from the wall 29 of the housing 22 towards the centerline 24. The restricting members 48 extend parallel to the wall 42 and are positioned closer to the second end 28 than the wall 42. The restricting members 48 are spaced from one another and spaced from the centerline 24. A gap or space 56 is formed between the restricting members 48 and the wall 42.

An opening 90 extends through the bottom (as shown) of the housing 22 into the microchannel 30 and between the walls 40, 42. The opening 90 can have any shape but regardless is used as a sample loading port by which blood samples can be injected or otherwise supplied to the microchannel 30, as will be described.

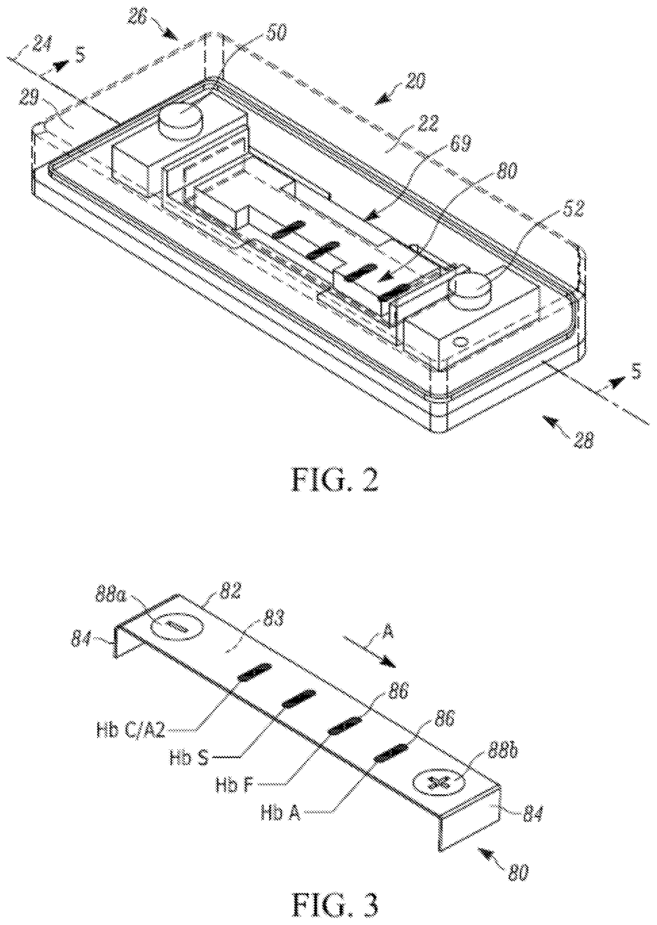

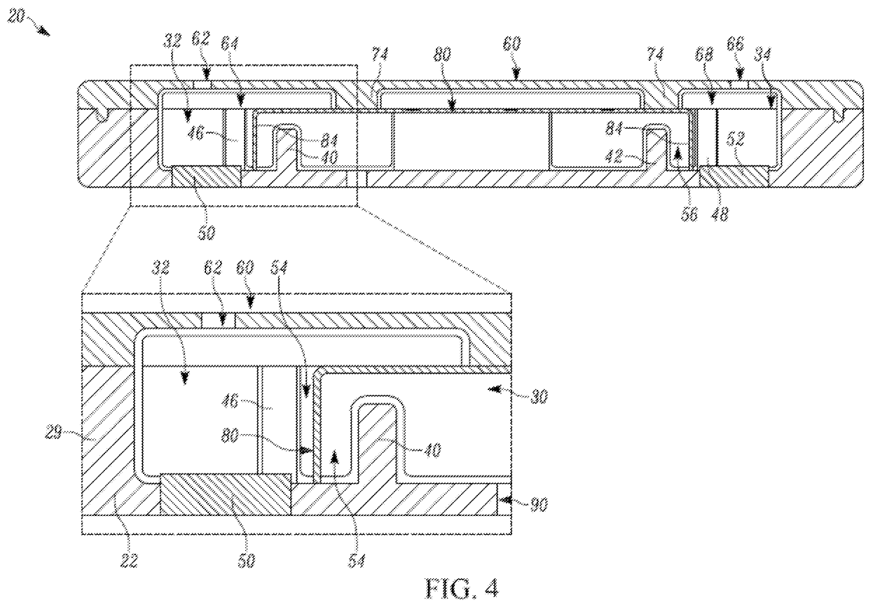

The indicating member 80 is elongated and includes a base 82 and a pair of legs 84 extending from the base. The indicating member 80 can be formed from the electrophoresis strip. Optionally, the electrophoresis strip 80 can be secured to or embedded in a hard, conductive material, e.g., metal, as indicated generally in phantom at 83 in FIGS. 1-2. In any case, the indicating member 80 is generally rectangular with the legs 84 extending at an angle, e.g., perpendicular, from the base 82. Consequently, the indicating member 80 can have a U-shaped construction.

A pair of electrode indications 88a, 88b is provided at opposite ends of the base 82. As shown, the indication 88a is a negative (−) terminal indication and the indication 88b is a positive (+) terminal indication. The terminal designations could, however, be reversed.

A series of test indications 86 can be provided along the base 82 between the electrode indications 88a, 88b and parallel to the length of the microchannel 30. Depending on the blood test to be performed, the test indications 86 can be symmetrically or asymmetrically spaced from one another along the base 82. The test indications 86 can be longitudinally aligned with one another or misaligned. The test indications 86 can have the same dimensions or different dimensions from one another. In one example, the test indications 86 are colored bands indicative of the basic types of hemoglobin, e.g., normal hemoglobin (HbAo), fetal hemoglobin (HbF), sickle hemoglobin (HbS), hemoglobin C (HbC or HbA₂), and non-glycosylated hemoblobin (HbA), and glycosylated hemoglobin (HbA1c).

The cover 60 is shaped similarly to the housing 22 and extends from a first end 61 to a second end 63. The cover 60 is generally rectangular and configured to be secured to the housing 22. The cover 60 can, for example, form a snap-fit connection with the housing 22. In any case, the cover 60 cooperates with housing 22 to define and enclose the microchannel 30.

A first opening 62 extends through the first end 61 and a second opening 66 extends through the second end 63. A first recess 64 is formed in the underside of the cover 60 and extends to the opening 64. A second recess 68 is formed in the underside of the cover 60 and extends to the opening 66.

The underside of the cover 60 further includes a plurality of support members 74 positioned between the openings 62, 66. As shown, the support members 74 are rectangular projections extending parallel to one another and perpendicular to the length of the cover 22.

A portion 69 of the housing 22 between the ends 26, 28 is transparent or optically clear to define an optical window that allows light to pass into and/or through a portion of the microchannel 30, e.g., the test indications 86 on the indicating member 80 when positioned within the microchannel. The ability to pass light can be a necessary step during analysis of a patient sample within the cartridge 20. The optical window 69 can be a material and/or construction that necessarily or desirably alters light entering the optical window 69 as a part of the analysis of the patient sample within, such as collimating, filtering, and/or polarizing the light that passes through the optical window 69. Alternatively, the optical window 69 can be transparent or translucent, or can be an opening within the housing 22 of the cartridge 20. The cartridge 20 can include a reflector (not shown) opposite the optical window 69 that reflects the incoming light back through the optical window 69 or through another optical window, or can include a further optical window opposite the light entry window to allow light to pass through the cartridge 20.

To this end, the portion or optical window 69 can optionally be provided with a hydrophilic coating to prevent spotting or hazing on the portion 69. The cover 60 and the housing 22 can both be formed from hard, durable materials, such as a plastic, polymer and/or glass.

When the device is assembled, the electrodes 50, 52 are positioned in the openings 36, 38 and extend into each buffer pool 32, 34. The legs 84 of the indicating member 80 are inserted into the gaps 54, 56 at each end 26, 28 of the housing 22 such that the indicating member 80 extends parallel to/along the housing centerline 24 within or adjacent to the microchannel 30. The restricting members 46, 48 and walls 40, 42 are longitudinally spaced from one another in a manner that prevents or limits longitudinal movement of the indicating member 80 relative to the housing 22. More specifically, the indicating member 80—in particular the electrophoresis strip, e.g., cellulose acetate paper—has a length substantially equal to the longitudinal distance between the restricting members 46, 48 such that the indicating member abuts the restricting members to prevent relative longitudinal movement therebetween.

The first buffer pool 32 and the second buffer pool 34 each receive a buffer solution 51, 53 that at least partially saturates the indicating member 80 extending into the respective pool. The buffer solution 51, 53 can exhibit an affinity to non-glycosylated hemoglobin, facilitate its separation from glycosylated hemoglobin, and thus be used for HbA1C testing.

In some embodiments, the buffer solution can be mildly acidic, for example, a pH of about 4.5 to about 6.7, (e.g., pH 6.4), and include a sulfated polysaccharide. The sulfated polysaccharide can bind to or exhibit an affinity to non-glycosylated hemoglobin. The sulfated polysaccharide is not particularly limited, and a known sulfated polysaccharide can be used. Specific examples include compounds for introducing a sulfate group to a neutral polysaccharide, such as cellulose, dextran, agarose, mannan or starch, or a derivative thereof, and salts of thereof; chondroitin sulfate; dextran sulfate; heparin; heparan; fucoidan; and the like. In certain embodiments, the sulfated polysaccharide can include dextran sulfate.

The buffer solution can also include organic acids such as citric acid, succinic acid, tartaric acid, and malic acid and salts thereof; amino acids such as glycine, taurine and arginine; inorganic acids, such as hydrochloric acid, nitric acid, sulfuric acid, phosphoric acid, boric acid and acetic acid, and salts thereof; and the like. Optionally, a generally used additive may be added to the above-mentioned buffer solution. Examples thereof include surfactants, various polymers, hydrophilic low-molecular-weight compounds, and the like.

By way of example, the buffer solution can include 33 mmol citrate, 2 μmol dextran sulfate, and 8 μmol disodium EDTA per liter, at a pH of 6.4. The buffer pools 32, 34 can each receive about 1 μL to about 200 μL of the respective buffer solution 51, 53.

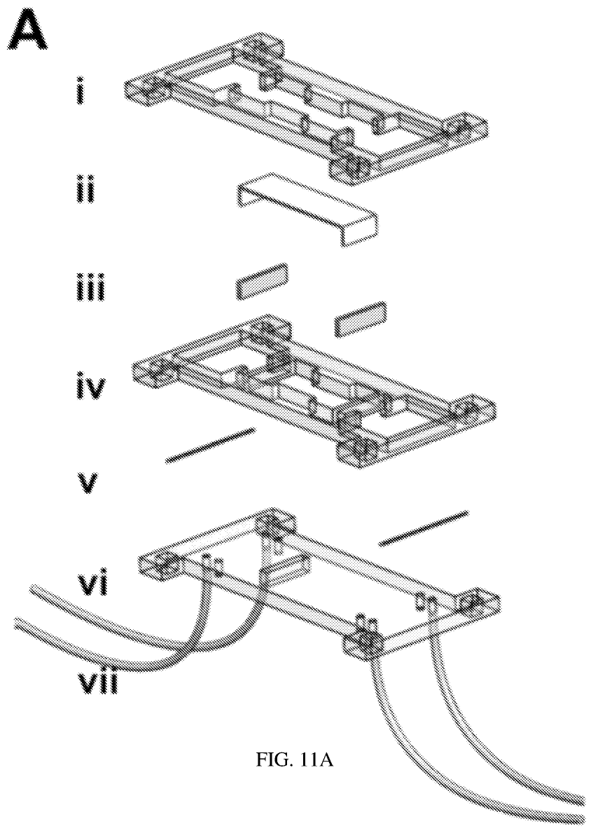

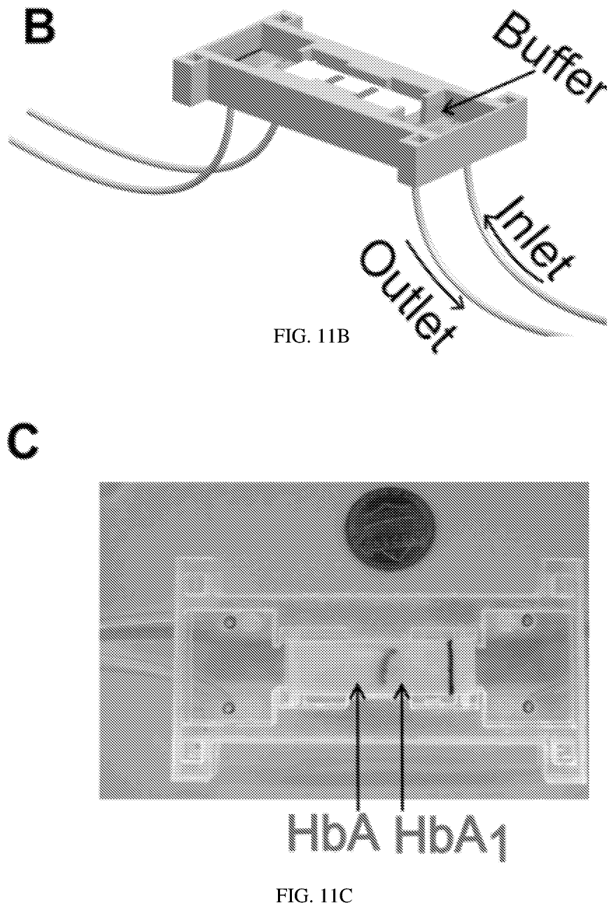

Optionally, as illustrated in FIG. 11, the system can include a buffer replenishing system configured to replenish the buffer solution for the first and second buffer pools and counteract pH changes in the buffer solution during operation of the system. The buffer replenishing system can include a first inlet conduit and a first outlet conduit in fluid communication with the first buffer pool and a second inlet conduit and a second outlet conduit in fluid communication with the second buffer pool for supplying and removing buffer solution from, respectively, the first buffer pool and the second buffer pool.

In some embodiments, the buffer solution can flow at a rate of about 3 μl/min to about 8 μl/min through the first inlet conduit and the second inlet conduit to the first buffer pool and the second buffer pool and at a rate of about 1 μl/min to about 3 μl/min from the first buffer pool and the second buffer pool through the first outlet conduit and the second outlet conduit.

The cover 60 is secured to the housing 22 to confine the indicator member 80 within the microchannel 30. The opening 62 in the cover 60 is aligned with the electrode 50 within the first buffer pool 32. The negative electrode indication 88a is generally positioned between the opening 62 and the electrode 50. The opening 66 in the cover 60 is aligned with the electrode 52 within the second buffer pool 34. The negative electrode indication 88b is generally positioned between the opening 66 and the electrode 52.

When the cover 60 is secured to the housing 22 the optical window 69 of the housing is aligned with the indicating member 80 such that all the test indications 86 on the electrophoresis strip are visible through the optical window 69. In other words, the support members 74 on the cover 60 do not visually obstruct the test indications 86 through the optical window 69.

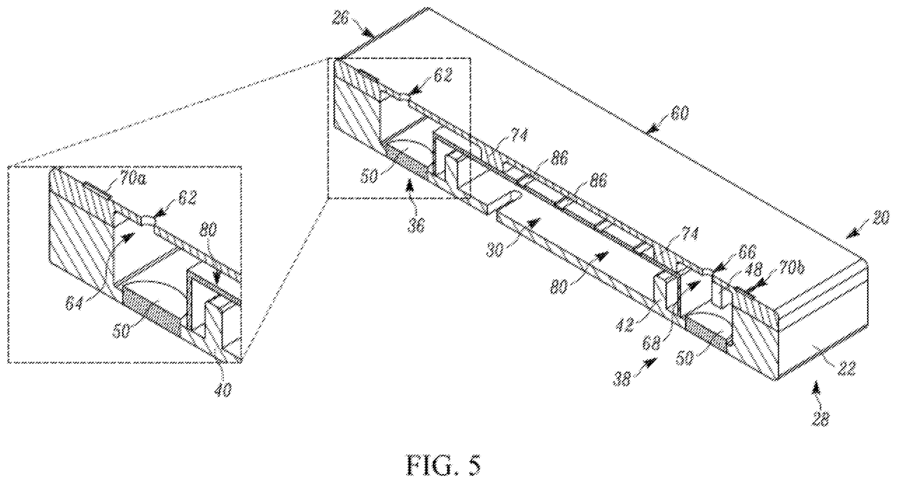

Referring to FIG. 5, an electrode 94 is inserted through the opening 62 in the cover, into the first buffer pool 32, and into contact with the electrode 50. An electrode 96 is inserted through the opening 66 in the cover, into the second buffer pool 34, and into contact with the electrode 52. The electrodes 94, 96 are electrically connected to a power supply 98. The electrodes 50, 52, 94, 96, buffer solutions 51, 53 and indicating member 80 cooperate to form an electrical circuit through the cartridge electrophoresis device 20.

The power supply 98 is capable of generating an electric field of about 1V to about 400V. In some instances, the voltage applied to the cartridge electrophoresis device 20 by the electrodes 94, 96 does not exceed 250V. Regardless, an electric field is generated across the electrophoresis strip of the indicating member 80 effective to promote migration of hemoglobin variants in a blood sample along the electrophoresis strip.

A patient sample, such as patient blood sample, can be provided in the cartridge 20 and on the electrophoresis strip using a sample applicator 92. The sample applicator 92 provides a more precise and/or controlled deposition of the sample onto the electrophoresis strip. Additionally, the patient sample can include added compounds/components, such as one or more markers. The added compounds/components can assist with the electrophoresis process and/or assist with interpreting the electrophoresis results.

For example, the one or more markers can have known migration rates and/or distances for a given applied voltage and/or voltage application time. Alternatively, these markers can normalize the results of the electrophoresis process by having migration rates relative to the sample, thereby reducing the effects of sample-to-sample variability. These markers can assist with evaluating the resultant banding of the patient sample.

A sample applicator 92 is filled with hemolysate of a blood sample BS and inserted into the loading port 90 on the underside of the housing 22. The hemolysate of the blood sample BS introduced into the loading port 90 can be, for example, less than about 10 μL. The cartridge electrophoresis device 20 can therefore be microengineered and capable of processing a small volume, e.g., a finger or heel prick volume.

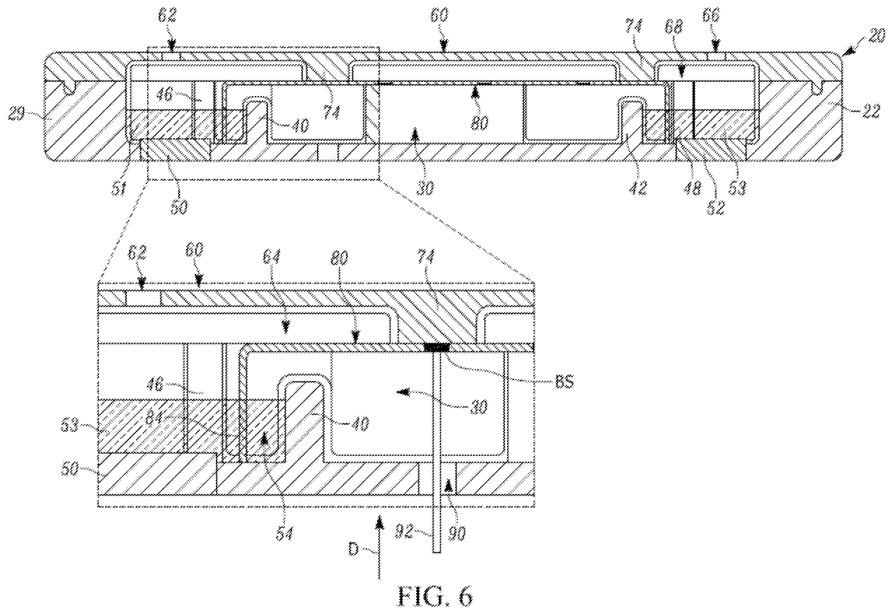

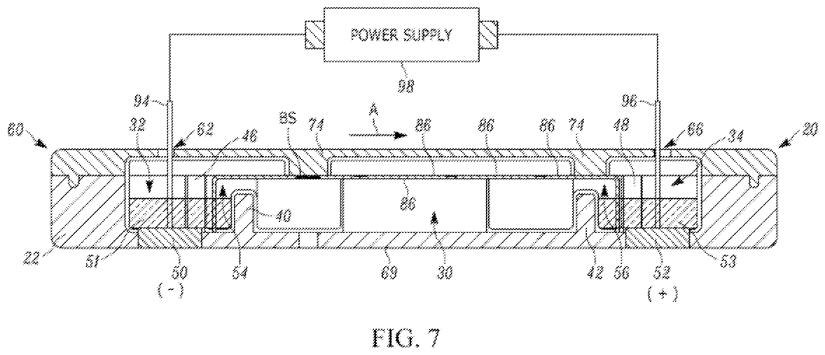

In any case, the sample applicator 92 is urged in the direction D towards the indicating member 80. The loading port 90 therefore helps to guide the sample applicator 92 towards a desired location on the indicating member 80. Since the indicating member 80 is formed from the electrophoresis strip, the sample applicator 92 can be inserted to the strip and release the sample BS therein to the left [as shown in FIG. 6] of all the test indications 86.

The loading port 90 is aligned with and extends towards one of the support structures 74. As a result, the support members 74—especially the leftmost support member—acts as a reaction surface to the indicating member 80 as the sample applicator 92 extends into the electrophoresis strip and deposits the sample therein. The support member 74 thereby prevents movement of the indicating member 80 away from the moving sample applicator 92. This helps prevent deformation or distortion of the indicating member 80 and helps the user release the sample in the proper location along the indicating member.

Once the sample BS is released in position, the sample applicator 92 is withdrawn (in a direction opposite D) from the electrophoresis device 20. Capillary action by the electrophoresis strip 80 can maintain the sample BS in position. The power supply 98 is actuated/turned on, which supplies current to the buffer pools 32, 34 and indicating member 80 as described, which establishes a continuous electrical path through the device 20 via the buffer pools 32, 34 and cellulose acetate paper 80 in contact therewith.

In this example, forming the indicating member 80 out of cellulose acetate paper allows the indicating member to also act as the sieving medium during the electrophoresis process. To this end, hemoglobin in the hemolysate of the blood sample BS are moved through the indicating member 80 and towards the positive electrode 52 in the direction indicated by A (FIG. 7).

With the patient sample in place, a voltage is applied using the electrodes, causing hemoglobin variant types to migrate across the electrophoresis strip over a defined time. The various hemoglobin types will separate into bands due to the applied voltage and the physical and electrical properties of the various hemoblobin types. One or all of the applied voltage, current and the application time can be predetermined or preset based on the various parameters of the electrophoresis testing being performed. Alternatively, one or more of the voltage, current and application times can be variable and based on the banding of the patient sample or an added compound/component therein. For example, the movement of a marker added to the patient sample can be monitored as the marker moves across the electrophoresis strip. That is, imaging/monitoring of the electrophoresis testing, and/or the markers thereon, can be performed in a continuous or timed interval manner during the testing process. For example, images of the electrophoresis process can be continuously captured, such as by a video imaging process, or the images can be captured at regular intervals based on time and/or the distance one or more bands have traveled. Once the marker has reached a predetermined location across the electrophoresis strip, the test can be terminated with the removal of the applied voltage.

After a predetermined time, the power supply 98 is turned off and the hemoglobin variants are frozen in their respective positions along the length of the indicating member 80 and relative to the various test indications 86. Depending on the distribution of the hemoglobin 86 a diagnosis regarding the sample BS can be made. To this end, the transparent portion 69 of the housing 22 allows the reader to simply visualize the distribution of hemoglobin relative to the test indications 86 on the indicating member 80. Consequently, a diagnosis regarding the sample BS can quickly be made. In the example shown, the user can identify different hemoglobin distributions within the hemolysate of the blood sample BS and readily diagnose any deficiencies or hemoglobin-related conditions.

The configuration of the cartridge electrophoresis device 20 is advantageous for several reasons. First, as noted, the support structures 74 provided on the cover 60 help prevent movement of the indicating member 80 while the sample BS is injected/provided into the cellulose acetate paper forming the indicating member. Second, the walls 40, 42 and associated restricting members 46, 48 each help prevent or limit relative movement between the indicating member 80 and the housing 22 during loading and operation of the device 20.

Moreover, the wall 40 also helps to prevent the buffer solution 51 within the first buffer pool 32 from leaking into the microchannel 30 via capillary action. Similarly, the wall 42 helps to prevent the buffer solution 53 within the second buffer pool 34 from leaking into the microchannel 30 via capillary action. The walls 40, 42 help ensure current flow through the device 20 is continuous and helps the device maintain a substantially constant pH during operation.

Additionally, embedding the electrodes 50, 52 within the bottom of the buffer pools 32, 34 helps to ensure a consistent supply of electric field through the cellulose acetate paper on the indicating member 80, even when/if either buffer solution 51, 53 begins to evaporate.

The reader 14 can include a housing (not shown) that surrounds and encloses some portion or all of the reader components. The housing of the reader 14 is constructed of materials, which may involve a suitably robust construction such that the reader 14 is rugged and portable. Alternatively, the reader 14 can be designed and/or constructed for use in a permanent or semi-permanent location, such as in a clinic or laboratory.

The housing of the reader 14 includes a cartridge interface that interacts with and/or engages the cartridge 12 for analysis of a patient sample. The cartridge interface can be a slot that is shaped to receive the cartridge 12. Alternative designs and/or structures of cartridge interfaces can be used with the reader 14.

The reader 14 can include an electrophoresis module 15 that can interface with the cartridge 12 to perform the electrophoresis test. The electrophoresis module 15, alone or in conjunction with processing circuitry, can control the electrophoresis test, including voltage/current application time and/or level. The electrophoresis module can supply electrical power from the power supply 98 to the cartridge 12, or electrophoresis strip, directly, to establish the necessary voltage across the electrophoresis strip for testing. The voltage can be applied at a higher level to increase the speed of the testing; however, the increased speed can cause decreased band fidelity, which can increase the difficulty and error of the band analysis and evaluation. A lower applied voltage can increase band fidelity but can lengthen the required testing time. Alternatively, the electrophoresis module 15 can vary the applied voltage or current, while maintaining the other stable, to achieve a desired or required level of band fidelity and testing speed. For example, an initial test to identify a patient condition can be carried out at a higher level voltage level to speed the test and a subsequent test to quantify the condition can be carried out a lower voltage level to generate clearer or more accurate results.

An electrophoresis band detection module 16, alone or in conjunction with the electrophoresis module 15, can capture, analyze and/or evaluate the electrophoresis test results and/or any other band detection characteristic(s) related to or otherwise based on the electrophoresis test results. The electrophoresis band detection module 16 can include a UV imaging system, such as a digital image sensor, to capture an image of the electrophoresis strip and the banding thereon at the conclusion of the electrophoresis test. Using the captured image data, each of the bands can be associated with one or more compounds/components of the patient blood sample and the proportions of each can be determined.

The UV imaging system is configured to illuminate the bands of non-glycosylated hemoglobin and glycosylated hemoglobin with UV radiation, detect through the optically transparent portion of the housing the bands of non-glycosylated hemoglobin and glycosylated hemoglobin on the electrophoresis strip caused by the applied electric field, and generate band detection data based on the bands of non-glycosylated hemoglobin and glycosylated hemoglobin. A processor can be configured to receive and analyze the band detection data to determine one or more band characteristics for the bands and generate diagnostic results based on the one or more band characteristics.

In some embodiments, the UV imaging system is configured to detect at least one of band size, migration, and intensity, and the processor includes a decision algorithm configured to generate density plots based on the band size, migration, and intensity and identify the relative percentages of glycosylated and non-glycosylated hemoglobin bands based on the generated density plots. For example, the processor can be configured to diagnose whether the subject has or is at risk of diabetes.

The reader 14 can also include an output 17 that includes one or more visual and/or audible outputs although in other examples the output is data and does not include visual and/or audible outputs. The output 17 communicates information regarding the status of the reader 14, the results of analysis of a patient sample, instructions regarding use of the reader 14 and/or other information to a user or other computing device. The output 17 can include a display, such as a screen, such as a touchscreen, lights, and/or other visual indicators. The touchscreen used to display information, such as analysis results, to the user can also be used by a user to input to the reader 14. Alternative interfaces can be included on and/or connected to the reader 14, such as a keyboard and/or mouse. Additionally, user devices, such as a cellphone or tablet, can be connected to the reader 14 to provide an interface portal through which a user can interact with the reader 14. The audible output 17 can include a speaker, buzzer, or other audible indicators. The output 17 can be output through an external device, such as a computer, speaker, or mobile device connected physically and/or wirelessly to the reader 14. The output 17 can output data, including the collected analysis data and/or interpretative data indicative of the presence or absence of a disorder, condition, infection and/or disease within the patient and/or the patient sample. An example can include the identification and proportions of the various hemoglobin types within the patient sample. The interpretive data output can be based on the analysis data collected and processed by the processing circuitry of the reader 14.

The reader can further include a sample processing module 18 or processor. The sample processing module 18 can receive inputs from the electrophoresis band detection module 16. Based on the received band detection data the sample processing module 16 can determine at least a characteristic of the patient sample, such as a disease or condition, an identity of the various compounds/components of the patient sample and quantification of the various compounds/components of the patient sample. The sample processing module 18 can output the identification and proportions of the compounds/components, and/or other various data based on the analysis of the patient sample. For example, the sample processing module 18, using the band detection data from the electrophoresis band detection module 16, can identify and quantify the various hemoglobin types of the patient sample. The output from the sample processing module 18 can be transmitted through the output 17 of the reader 14 or transmitted to an external device and/or system, such as a computer, mobile device, and remote server or database.

The sample processing module 18 can analyze the patient sample to determine a hemoglobin characteristic, such as a hemoglobin affecting disease and/or condition, based on the data from various components, elements and/or systems of the reader 14. The results of the analysis can be output from the sample processing module 18 to the output 17 to convey the information to a user or other.

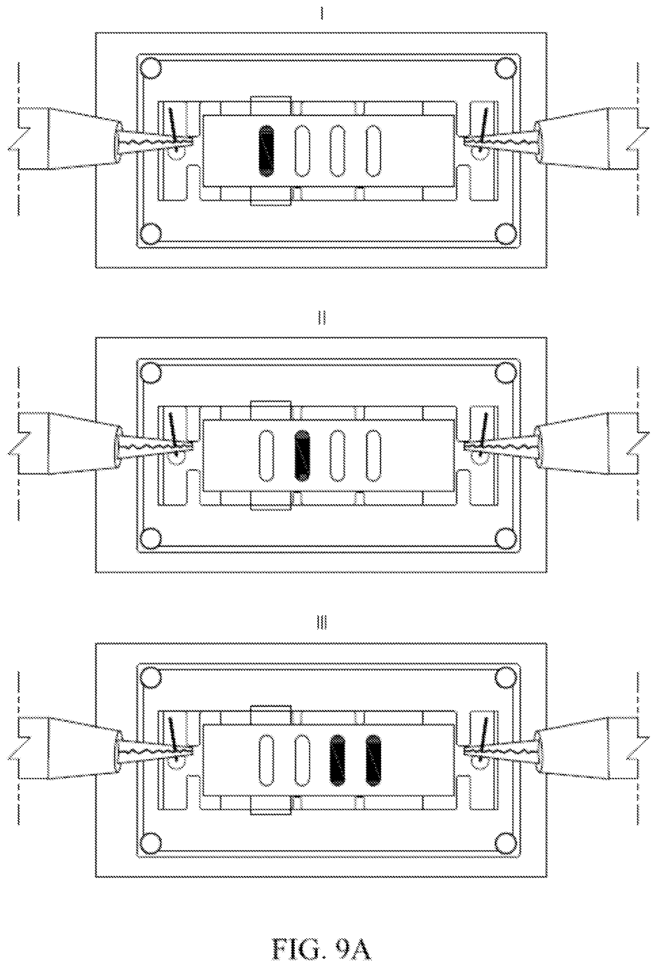

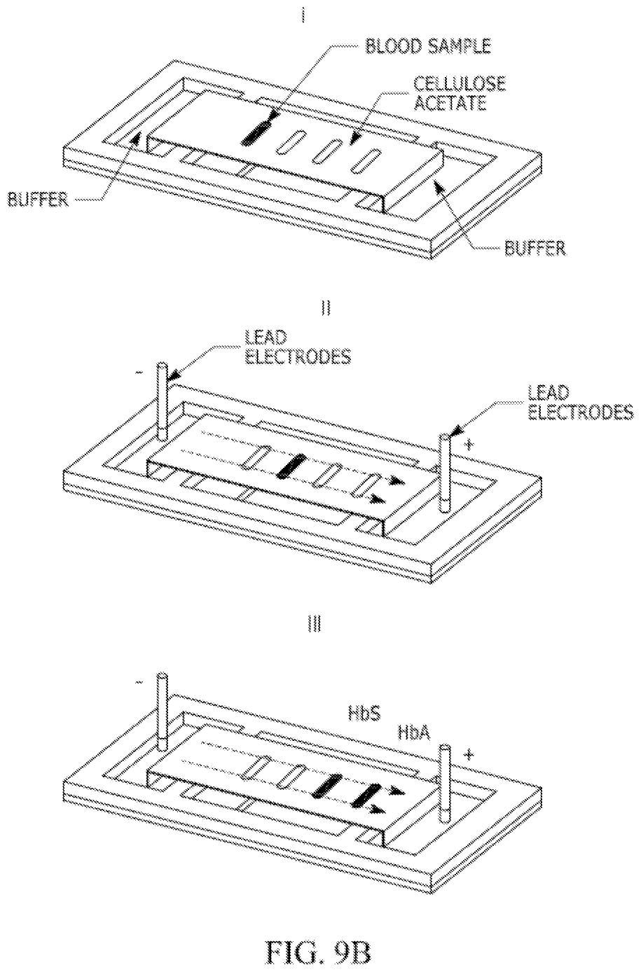

Referring to FIGS. 9(A-B), another example of a cartridge electrophoresis device or 200 can include a housing having first and second buffer ports, a sample loading port, and first and second electrodes. The housing also includes a microchannel that extends from a first end to a second end of the housing. The microchannel contains an electrophoresis strip (e.g., cellulose acetate paper) that is at least partially saturated with a buffer solution that exhibits an affinity to non-glycosylated hemoglobin, which facilitates its separation from glycosylated hemoglobin, and can thus be used for HbA1C testing. The first buffer port and the second buffer extend, respectively, through the first end and second end of the housing to the microchannel and electrophoresis strip. The first buffer port and the second buffer port are capable of receiving the buffer solution that at least partially saturates the electrophoresis strip.

The sample loading port can receive hemolysate of a blood sample and extends through the first end of the housing to the microchannel and cellulose acetate paper. The first electrode and the second electrode can generate an electric field across the electrophoresis effective to promote migration of hemoglobin variants in the hemolysate of the blood sample along the cellulose acetate paper. The first electrode and second electrode can extend, respectively, through the first buffer port and the second port to the electrophoresis strip.

In some embodiments, the housing can include a top cap, a bottom cap, and a channel spacer interposed between the top cap and the bottom cap. The channel spacer can define the channel in the housing. The top cap, bottom cap, and channel spacer can be formed from at least one of glass or plastic.

In some embodiments, the diagnostic system can further include a reader that includes an imaging system for visualizing and quantifying hemoglobin variant band migration along the electrophoresis strip for blood samples introduced into the sample loading port. The housing can include a viewing area for visualizing the cellulose acetate paper and hemoglobin variant migration.

The first electrode and the second electrode can be connected to a power supply. The power supply can generate an electric field of about 1V to about 400V. In some embodiments, the voltage applied to the cartridge by the electrodes does not exceed 250V.

The cartridge can be microengineered and be capable of processing a small volume (e.g., for example, a fingerprick volume or a heelprick volume).

The sample can be a blood sample that can be optionally treated, if necessary or desired, for analysis. The treatment of the blood sample can include diluting the blood sample, which can be done by mixing the collected blood sample with a dilutant, such as deionized water or other fluid that dilutes the blood sample. The dilutant can alter the viscosity of the blood sample, the opacity or translucence of the blood sample, or otherwise prepare the blood sample for analysis using the reader. Preferably, the dilutant does not impact the resulting analysis of the blood sample and/or assists with preparing the blood sample for analysis. This can include lysing the cells of the blood sample to release the various cellular components for electrophoresis analysis by the reader. Lysing agents can include fluids, such as water or various chemicals, and powders. Additionally, mechanical lysing can be used, such as by sonication, maceration and/or filtering, to achieve adequate lysing of the cells of the blood sample in preparation for analysis of the sample.

One or more markers can be added to the blood sample. The added markers can assist with visualizing the completed electrophoresis results. For example, a marker that moves at the same relative rate as a hemoglobin type due at a predetermined applied voltage can be added. The marker will move with the hemoglobin type containing portion of the blood sample across the electrophoresis strip in response to the applied voltage. The marker can have a color, or other optical properties that makes visualizing the marker easier. Since the marker moves with or relative to a specific hemoglobin type, the easier to visualize marker can make it easier to determine the distance the hemoglobin type has moved across the electrophoresis strip in response to the applied voltage.

In other embodiments, the sample introduced into the sample loading port can be less than 10 μL. The buffer solution can include alkaline tris/Borate/EDTA buffer solution. The first electrode and the second electrode can include graphite or carbon electrodes.

In some embodiments, the diagnostic system can be used to diagnose whether the subject has hemoglobin variants HbAA, HbSS, HbSA, HbSC, HbA2, HbA1, and HbA1c. In other embodiments, the diagnostic system can be used to diagnose whether the subject has or an increased risk of diabetes.

In some embodiments, the diagnostic system can be used in a method where hemolysate of a blood sample from a subject is introduced into the sample loading port. The blood sample includes hemoglobin. Hemoglobin bands formed on the cellulose acetate paper are then imaged with the imaging system to determine hemoglobin phenotype for the subject. The hemoglobin phenotype can be selected from the group consisting of HbAA, HbSA, HbSS, HbSC, HbA2, HbA1, and HbA1c.

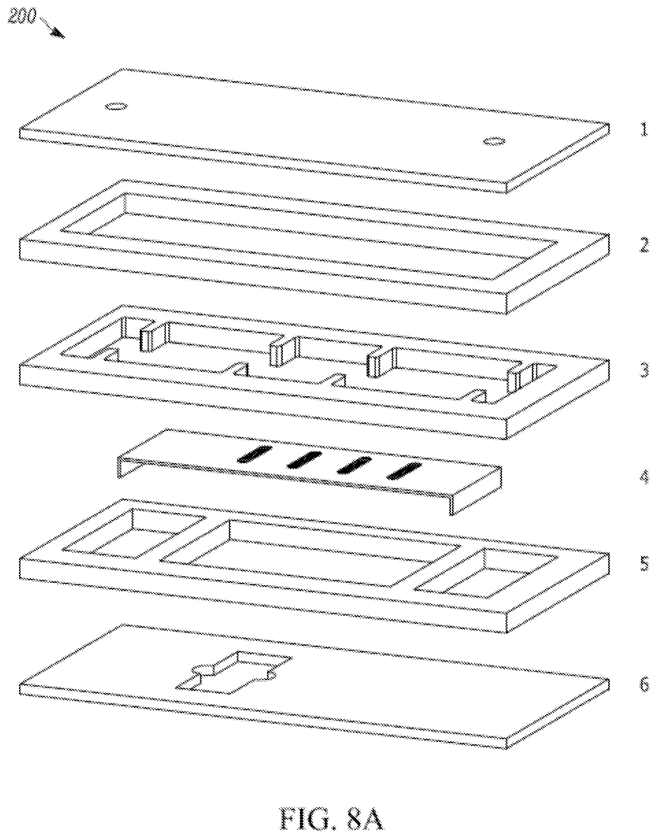

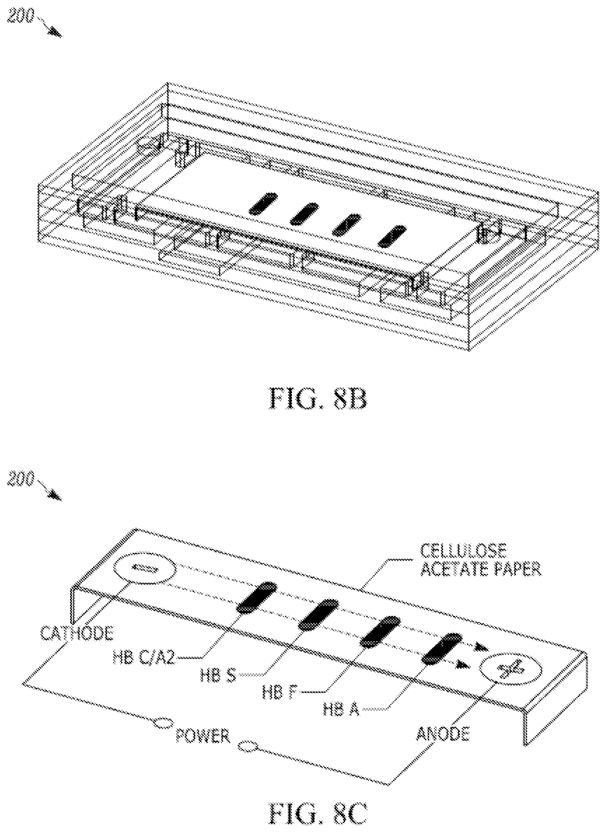

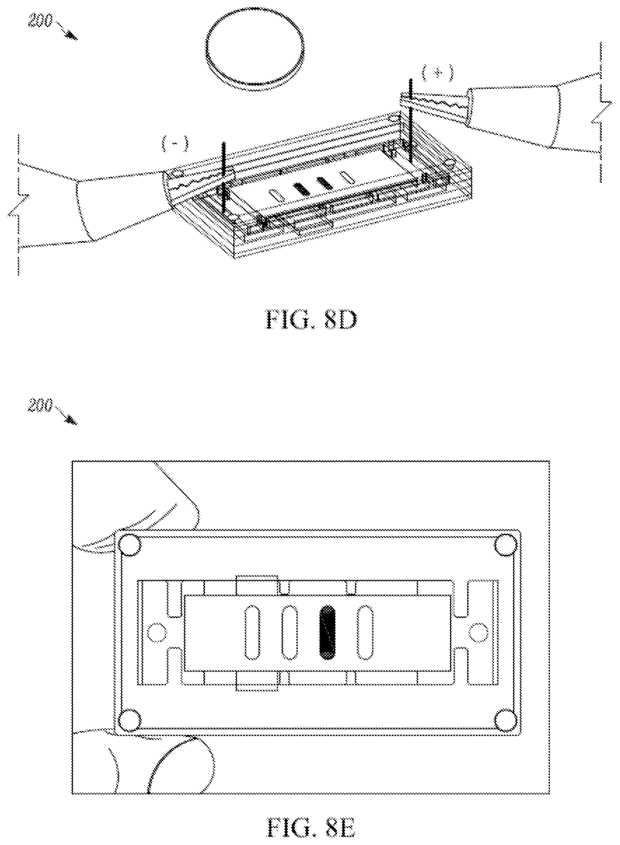

In some embodiments, the cartridge comprises biomedical grade poly methyl methacrylate (PMMA, McMaster-Carr) substrates and a double sided adhesive film (DSA)(3M Company), which have been shown to be biocompatible and non-cytotoxic in biomedical and clinical applications. Cartridges may be fabricated using a micromachining platform (e.g., X-660 Laser, Universal Laser Systems) to create a variety of structures including, but not limited to, inlet ports, outlet ports, sample ports, microfluidic channels, reaction chambers, and/or electrophoresis channels. (FIG. 8A) Microfluidic channel dimensions may be controlled to within 10 μm. In other embodiments, the diagnostic system allows rapid manual assembly and is disposable (e.g., for example, a single use cartridge) to prevent potential cross-contamination between patients.

One advantage of the diagnostic system described herein, and particularly, the cartridge electrophoresis device, is that it is suitable for mass-production which provides efficiency in point-of-care technologies. The diagnostic system can provide a low cost screen test for monitoring glucose levels and diabetes in a subject. It is mobile and easy-to-use; it can be performed by anyone after a short (30 minute) training. The diagnostic system described herein can integrate with a mobile device (e.g., IPhone, IPod) to produce objective and quantitative results. If necessary, cartridge electrophoresis devices and/or their components may be sterilized (e.g., by UV light) and assembled in sterile laminar flow hood. Sterile biomedical grade silicon tubing (Tygon Biopharm Plus) may be integrated to the cartridge electrophoresis devices and cartridge electrophoresis devices may be sealed to prevent any leakage. Further, tubing allows simple connection to other platforms, such as in vitro culture systems for additional analyses if needed.

In other embodiments, a mobile imaging and quantification algorithm can be integrated into the diagnostic system and/or reader. The algorithm can achieve reliable and repeatable test results for data collected in all resource settings of the diagnostic system.

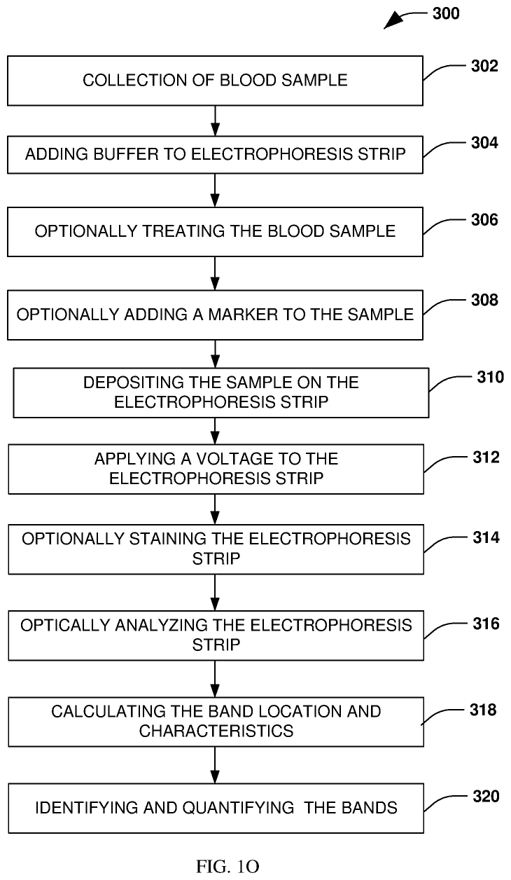

FIG. 10 is an example analysis method 300 identifying and quantifying glycosylated hemoglobin (HbA1c) and non-glycosylated hemoglobin (Hba). The analysis of a patient sample, which is patient blood in this example, is performed to determine average blood glucose concentration over a period of two months preceding the taking of a blood sample. The example method of FIG. 10 can be performed using a reader and cartridge, such as the example shown in FIG. 1. The reader can include one or more systems and/or elements to analyze, quantify, identify and/or otherwise determine HbA1c characteristics of a patient sample that can be indicative of the presence of diabetes in the patient.

An initial step 302 of the method 300 can include the collection of a patient sample for analysis, in this example, a blood sample.

At 304, a buffer can be added to the electrophoresis strip in preparation for the electrophoresis testing of the collected blood sample. The buffer solution can exhibit an affinity to non-glycosylated hemoglobin, facilitate its separation from glycosylated hemoglobin, and thus be used for HbA1C testing.

In some embodiments, the buffer solution can be mildly acidic, for example, a pH of about 4.5 to about 6.7, (e.g., pH 6.4) and include a sulfated polysaccharide. The sulfated polysaccharide can bind to or exhibit an affinity to non-glycosylated hemoglobin.

The sulfated polysaccharide is not particularly limited, and a known sulfated polysaccharide can be used. Specific examples include compounds for introducing a sulfate group to a neutral polysaccharide, such as cellulose, dextran, agarose, mannan or starch, or a derivative thereof, and salts of thereof; chondroitin sulfate; dextran sulfate; heparin; heparan; fucoidan; and the like. In certain embodiments, the sulfated polysaccharide can include dextran sulfate.

The buffer solution can also include organic acids, such as citric acid, succinic acid, tartaric acid, and malic acid and salts thereof; amino acids, such as glycine, taurine and arginine; inorganic acids, such as hydrochloric acid, nitric acid, sulfuric acid, phosphoric acid, boric acid and acetic acid, and salts thereof; and the like. Optionally, a generally used additive may be added to the above-mentioned buffer solution. Examples thereof include surfactants, various polymers, hydrophilic low-molecular-weight compounds, and the like.

By way of example, the buffer solution can include 33 mmol citrate, 2 μmol dextran sulfate, and 8 μmol disodium EDTA per liter, at a pH of 6.4.

The collected blood sample 302 can then be treated at step 306, if necessary or desired, for analysis. The treatment of the blood sample can include diluting the blood sample, which can be done by mixing the collected blood sample with a dilutant, such as deionized water or other fluid that dilutes the blood sample. The dilutant can alter the viscosity of the blood sample, the opacity or translucence of the blood sample, or otherwise prepare the blood sample for analysis using the reader. Preferably, the dilutant does not impact the resulting analysis of the blood sample and/or assists with preparing the blood sample for analysis. This can include lysing the cells of the blood sample to release the various cellular components for electrophoresis analysis by the reader. Lysing agents can include fluids, such as water or various chemicals, and powders. Additionally, mechanical lysing can be used, such as by sonication, maceration and/or filtering, to achieve adequate lysing of the cells of the blood sample in preparation for analysis of the sample.

At 308, one or more markers can be optionally added to the blood sample. The added markers can assist with visualizing the completed electrophoresis results. For example, a marker that moves at the same relative rate as a hemoglobin type due at a predetermined applied voltage can be added. The marker will move with the hemoglobin type containing portion of the blood sample across the electrophoresis strip in response to the applied voltage. The marker can have a color, or other optical properties that makes visualizing the marker easier. Since the marker moves with or relative to a specific hemoglobin type, the easier to visualize marker can make it easier to determine the distance the hemoglobin type has moved across the electrophoresis strip in response to the applied voltage.

At 310 the blood sample can be deposited onto the electrophoresis strip in a controlled manner, preferably applied in a “line” perpendicular to the length of the electrophoresis strip. The controlled manner of deposition can include controlling the amount of blood sample deposited, the area across which the blood sample is deposited, the shape of the area across which the blood sample is deposited and/or other deposition characteristics. One or more systems and/or components of the reader and/or cartridge can be used to deposit the blood sample in the controlled manner onto the electrophoresis strip.

With the blood sample deposited onto the electrophoresis strip, a voltage can be applied across the electrophoresis strip at 312 to cause the separation of the blood sample into various bands of glycosylated hemoglobin and non-glycosylated hemoglobin. The voltage or current can be applied at a predetermined level or series of levels and for an amount of time. As discussed previously, the application time of the voltage can be predetermined or based on the movement of one or more bands of the patient sample, measurement of an electrical parameter such as resistance or an added compound/component. A higher applied voltage can cause the bands to move across the electrophoresis strip at a greater speed, however, the band shape can be distorted making the interpretation of the banding difficult. A lower applied voltage can increase band fidelity but can take a longer time to perform the requisite testing. The applied voltage can be selected to optimize testing efficiency while maintaining a desired or minimum fidelity level. Further, the applied voltage can be varied during testing, such as applying a higher voltage initially and then applying a lower voltage. The varied application of the voltage can cause the initial band separation and movement and the later applied lower voltage can assist with increasing the fidelity of the resultant banding pattern. Additionally, varying voltages and/or currents can be applied during the electrophoresis process in response to a measurement of the bands formed by the blood and/or the band or bands formed by the markers in a predetermined ratio, to maintain a constant rate of travel of the marker band or a portion thereof.

After completion of the electrophoresis process, the electrophoresis strip can be optionally stained at 314. Staining the electrophoresis strip and the bands thereon can assist with the analysis and/or evaluation of the banding. For example, a stain for hemoglobin can be used to stain the bands to assist with determining a position of the bands across the electrophoresis strip. The cartridge and/or reader can include the stain and the required systems/components for applying the stain to the electrophoresis strip. Alternatively, a user can stain the electrophoresis strip before band analysis. Alternatively or in addition, a short high voltage can be applied at the end of the test essentially burning the hemoglobin bands and making them visually persistent. The high voltage may also reduce the risk of viable pathogens.

At 316, the electrophoresis strip can be optically analyzed, for example, by UV imaging the electrophoresis strip and the bands thereon. Data collected under UV light illumination is then used for sensitive and accurate identification and quantification of glycosylated hemoglobin. For example, data collected under UV illumination with 410 nm illumination has a greater limit of detection and a higher signal-to-background ratio, as hemoglobin protein has a strong optical absorption around 414 nm. Using UV detection, the system can automatically track, detect, identify, and quantify electrophoretically separated low concentration HbA1. The image capture can be accomplished using one or more imaging sensors, such as a digital imaging sensor and can be performed throughout the testing process or at the conclusion of the test. With the UV imaging system, all bands appear dark; however, bands are identified depending on their size and intensity. The captured image(s) can be processed to evaluate and/or analyze the electrophoresis test results.

At 318, the final location and characteristics of the bands can be calculated. The calculation can determine the distance each of the bands traveled, due to the applied voltage during testing, from the initial blood sample placement on the electrophoresis strip as well as the area of the bands. Along with the distance of travel, a speed of travel of each band can be calculated based on the elapsed voltage application time and the distance traveled. Using the identity, area, and location of each of the bands, the various components/compounds of the initial blood sample, and their proportions, can be determined.

As part of the analysis of the electrophoresis tests, the bands formed during the testing can be identified and quantified at 320. Identification of the bands can include associating one or more compounds/components of the initial blood sample with each of the bands of the electrophoresis. For example, image processing and a decision algorithm can generate density plots based on the size, migration, and intensity patterns to identify the relative percentages of glycosylated and non-glycosylated hemoglobin bands. The identification of the bands can be assisted by markers that were previously added to the blood sample prior to the electrophoresis testing. The markers can be selected so that their final position along the electrophoresis test aligns with one or more of the compounds/components of interest in the blood sample. Alternatively, the marker can be selected to be interspersed between two bands so assist with differentiating the bands for identification.

Once the analysis of the blood sample is complete, the results can be output. The output of the results can include the identified and quantified HbA1c relative to non-glycosylated hemoglobin, which can be indicative of diabetes. The output can be displayed or relayed to the user in a visual output, such as on a display, auditory such as by a speaker, or other manner. This can include transmitting the output results to an external device, such as a computer, through a wired or wireless connection or communication protocol, such as by a Bluetooth connection.

### EXAMPLE

### Materials and Methods

The HemeChip-GHb utilizes 1×Glyco-Phore buffer at pH 6.5, an affinity electrophoresis buffer solution reconstituted from a 10×Glyco-Phore buffer solution made from 33 Mm/L citrate, 2 μM dextane sulfate and 8 μM EDTA per 1L of, deionized (DI) water (MilliQ, Academic, Billerica, MA). RBCs are washed with Sodium chloride solution prepared at 8.5 g/l, and then hemolyzed using a lysing buffer prepared from 1 g of saponin mixed with EDTA per liter.

There are five compartments in the HemeChip-GHb complete cartridge. Cartridge and tygon tubing, cellulose acetate sheets, blotting pads, horizontal platinum thin electrodes (FIG. 11A-C). Plastic backed cellulose acetate paper was purchased from Med-Tex (Philadelphia, PA). Blotter pads were purchased from Helena laboratories, Inc (Beaumont, Texas). Thin platinum electrodes (wire) of diameter 0.05 mm were purchased from Good Fellow (Coraopolis, PA). The cartridge was designed using lamination based fabrication method from plastic parts made from Optix CA-41 polymethyl Methacrylate Acrylic (PMMA) sheets from McMaster-Carr(Elmhurst, IL) and ePlastics (San Diego, CA) that were laser cut (VersalLaser VL) and laminated with 3M optically clear double sided adhesive (DSA) purchased from iTapeStores. Tygon tubing were purchased from Cole Parmer (Vernon, Illinois) (FIG. 11A). The cartridge was manually connected to an external power source of 70 V, 3A through the platinum electrode. The newly designed glycosylated hemoglobin microchip cellulose paper-based electrophoresis test cartridge allowed for real-time glycosylated hemoglobin monitoring and quantitative analysis (FIG. 11D).

HemeChip-GHb testing consists of three steps: (i) test preparation, (ii) sample preparation, (iii) and hemoglobin separation. To prepare for the HemeChip-GHb testing, the cellulose acetate membrane and blotting pad are socked with 1×Glyco-Phore buffer. 10 μl of lysed RBCs were then loaded into the applicator wells, this step allowed for only the required sample to be loaded to the micro-applicator. 8 μl of the sample from the applicator wells was then loaded into a capillary based micro-applicator. The micro-applicator was designed using metal lancet and PMMA sheet which are spaced by 150 μm. Then sample from the micro-applicator is loaded onto the wetted cellulose acetate strip and the strip is transferred into the cartridge. The electrodes are connected to an external bench top power supply set at 70 V, 3 A. Finally, prior to starting the test, 200 μl of Glyco-Phore buffer are loaded into the buffer ports and then the inlet and outlet tygon tubing are connected to the buffer replenishing system. The inlet buffer flows at rate of 4 μl/min and the outlet at 2 μl/min for both buffer ports. The test runs for 20 min to achieve complete separation of the band.

During testing, the cartridge is mounted on top of a chamber designed with an LED array (410 nm). LEDs with 410 nm illumination wavelength were used to leverage hemoglobin's highest optical absorption around 414 nm. The power supply to the LED array was set at 6V, to maintain a constant output intensity from LEDs during testing. A camera was used to obtain live time-lapse images of the cartridge during a test run. The camera was mounted on a fixed stand above the cartridge that was fixed on top of the UV chamber. Camera was set to manual, at F/16, focal length 40.0 mm, ISO-400, exposure time ½ sec and no flash mode. The camera was connected to Digi-cam control software V12 installed on a computer though a USB cable. In the Digi-cam control software the camera was set to live and every 10 s, an image frame was captured from the testing cartridge for the 20 min (FIG. 2A).

A customized data analysis algorithm was developed using python 3.8. This algorithm automatically identified HbA₁ and HbA based on glycosylated hemoglobin derivative band migration patterns. 74 UV image frames taken during the test run were uploaded into the algorithm. The algorithm then automatically quantified the relative percentages of HbA₁ and HbA. The reported percentages of HbA₁ and HbA from the glycosylated microchip electrophoresis were then compared to the calculated HPLC results using One-Way Mann Whitney test. The reproducibility of the glycosylated microchip electrophoresis was tested using normal, prediabetic and diabetic blood samples. We performed the reproducibility test using 3 samples at the different glycosylated hemoglobin levels. Each sample was tested 3 times using HemeChip GHb and compared against HPLC reference standard ranges of 6.64% (<6.77%-6.89, healthy), 14.02% (15.6-16.3%, diabetic).

### Results

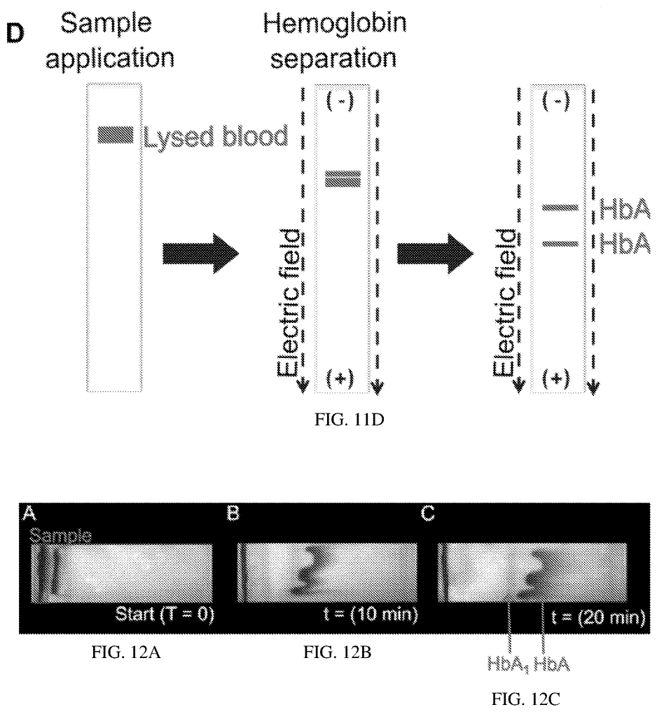

HemeChip was developed as the first miniaturized fully integrated single-use cartridge based microchip electrophoresis device for the detection and quantification of hemoglobin variants. HemeChip technology offers a timely, original and innovative solution, leveraging a novel engineering approach, to POC diagnosis of hemoglobinopathies (FIGS. 11A-E). HemeChip separates hemoglobin protein types in a minute volume of blood on a piece of cellulose acetate paper that is housed in a microengineered chip with a controlled environment and electric field (FIG. 11A-C). Differences in hemoglobin mobilities allow separation to occur within the cellulose acetate paper. The basis of HemeChip technology lies in hemoglobin electrophoresis in which hemoglobin types such as, A (normal), S (sickle), C (hemoglobin C disease), A2 (βthal, thalassemia), Bart's, and F (fetal) have net negative charges in an alkaline solution. Tris/Borate/EDTA (TBE) buffer is used to provide the necessary ions for electrical conductivity at a pH of 8.3. The overall negative net charges of the hemoglobins cause them to travel toward the positive electrode when placed in an electric field (FIG. 11C). Differences in hemoglobin mobilities allow separation to occur within the sieving medium, cellulose acetate. HemeChip is able to evaluate all the same variants as cellulose acetate electrophoresis, which is a standard test currently used in screening for hemoglobin disorders. One variation of cellulose acetate electrophoresis called affinity electrophoresis is used for the detection of glycosylated hemoglobin. This method utilizes an acidic buffer (pH 6.4) that exhibits an affinity to non-glycosylated hemoglobin, which facilitates its separation from glycosylated hemoglobin (FIG. 11D and FIGS. 12(A-B)), and can thus be used for A1C testing.

HemeChip-GHb technology is based on mobile affinity electrophoresis. The 1×Glyco-Phore buffer at pH 6.5 used contains dextran sulfate, a low molecular mass active ligand that binds to non-glycosylated hemoglobin and provides an additional negative charge, allowing electrophoretic separation of glycosylated hemoglobin and non-glycosylated hemoglobin. During the separation of non-glycosylated hemoglobin from glycosylated hemoglobin, the band is imaged using ultraviolet (UV) light. Data collected under UV light illumination is then used for sensitive and accurate identification and quantification of glycosylated hemoglobin. Data collected under UV illumination with 410 nm illumination has a greater limit of detection and a higher signal-to-background ratio, as hemoglobin protein has a strong optical absorption around 414 nm. Using UV detection, HemeChip-GHb automatically tracks, detects, identifies, and quantifies electrophoretically separated low concentration HbA1.

The HemeChip-GHb employs a real-time image tracking and data analysis approach. The HemeChip-GHb algorithm recognizes the initial lysate sample application and monitors the glycosylated hemoglobin and non-glycosylated hemoglobin separation until the end of the test (FIG. 12). With the UV imaging system, all bands appear dark; however, bands are identified depending on their size and intensity. Image processing and a decision algorithm generate density plots based on the size, migration, and intensity patterns to identify the relative percentages of glycosylated and non-glycosylated hemoglobin bands.

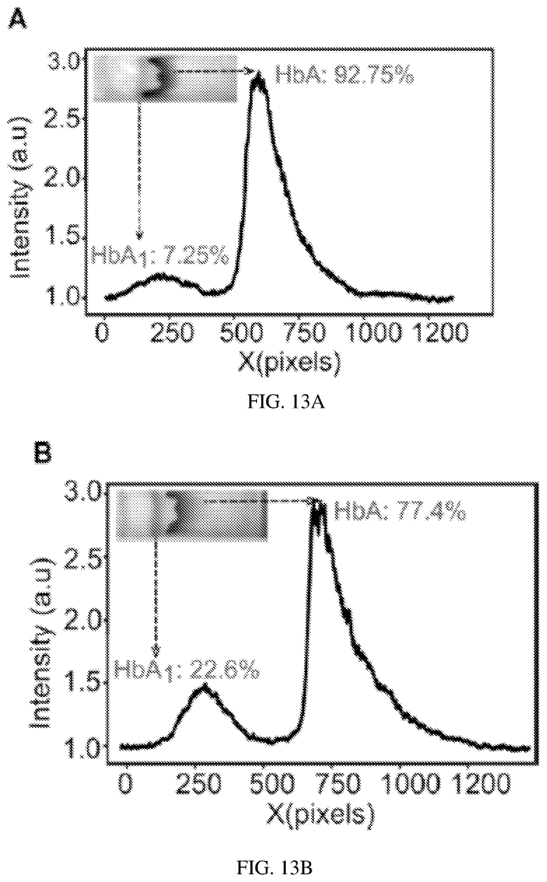

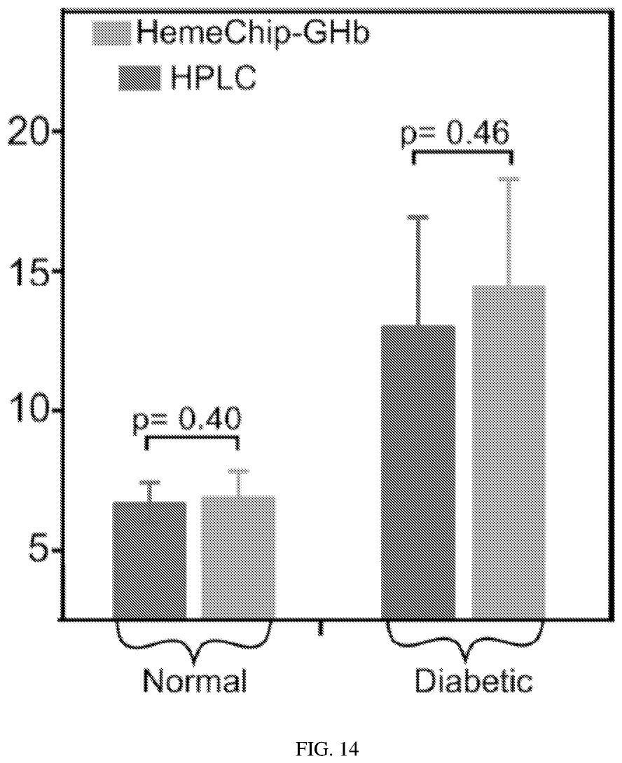

Two representative tests with different HbA1 were demonstrated in (FIG. 13A): Healthy sample; (FIG. 13B): diabetic levels. HbA1 measured levels for normal (n=7) and diabetic (n=5) patients were found to be statically significate to HbA₁ levels calculated from the reference standard method (HPLC) results (FIG. 14). Our results demonstrate no statistically significant difference, and hence a close agreement between HemeChip-GHb and the reference standard method (one-way Mann Whitney; Normal samples p=0.40 and diabetic p=0.46). These results reveal a strong association between GHb HbA1 levels determined by HemeChip and HbA₁ levels reported by HPLC.

HemeChip-GHb technology offers an original and innovative solution to POC diagnosis and monitoring of diabetes. In less than 20 minutes, the HemeChip-GHb prototype can process, analyze and display HbA1 results. This platform enabled the identification and measurement of HbA1 from HbA.

From the above description of the invention, those skilled in the art will perceive improvements, changes and modifications. Such improvements, changes, and modifications are within the skill of the art and are intended to be covered by the appended claims. All patents and publications identified herein are incorporated by reference in their entirety.

1. A diagnostic system, comprising:
a) a cartridge that includes:
a housing including a microchannel extending between first and second buffer pools each containing a buffer solution, the buffer solution exhibiting an affinity to non-glycosylated hemoglobin, which facilitates its separation from glycosylated hemoglobin;
an electrophoresis strip positioned within the microchannel and structured to receive at least a portion of a hemolysate, the electrophoresis strip having first and second ends positioned in the first and second buffer pools so as to be at least partially saturated with the buffer solution in each buffer pool; and
a first electrode connected to the housing and exposed to the buffer solution in the first buffer pool and a second electrode connected to the housing and exposed to the buffer solution in the second buffer pool, the first and second electrodes configured to generate an electric field across the electrophoresis strip, the application of the electric field to the first and second electrodes inducing migration and separation of bands of non-glycosylated hemoglobin and glycosylated hemoglobin in the hemolysate delivered to the electrophoresis strip; wherein a portion of the housing is optically transparent for visualizing the migrated and separated bands of non-glycosylated hemoglobin and glycosylated hemoglobin on the electrophoresis strip; and
b) a UV imaging system configured to illuminate the bands of non-glycosylated hemoglobin and glycosylated hemoglobin with UV radiation, detect through the optically transparent portion of the housing the bands of non-glycosylated hemoglobin and glycosylated hemoglobin on the electrophoresis strip caused by the applied electric field; and generate band detection data based on the bands of non-glycosylated hemoglobin and glycosylated hemoglobin.

2. The diagnostic system of claim 1, wherein UV imaging system includes a UV source that illuminates the bands with UV radiation at a wavelength of about 400 nm to about 420 nm.

3. The diagnostic system of claim 1, further comprising a processor configured to receive and analyze the band detection data to determine one or more band characteristics for the bands and generate diagnostic results based on the one or more band characteristics.

4. The diagnostic system of claim 3, wherein the UV imaging system is configured to detect at least one of band size, migration, and intensity, and the processor includes a decision algorithm configured to generate density plots based on the band size, migration, and intensity and identify the relative percentages of glycosylated and non-glycosylated hemoglobin bands based on the density plots.

5. The diagnostic system of claim 4, wherein the processor is configured to diagnose whether the subject has or is at risk of diabetes.

6. The diagnostic system of claim 1, wherein the buffer solution is acidic and includes a sulfated polysaccharide.

7. The diagnostic system of claim 1, further comprising a power supply connected to the first electrode and the second electrode configured to supply the electric field across the electrophoresis strip.

8. The diagnostic system of claim 1, further comprising a buffer replenishing system configured to replenish the buffer solution for the first and second buffer pools and counteract pH changes in the buffer solution during operation of the system.

9. The diagnostic system of claim 8, wherein the buffer replenishing system includes a first inlet conduit and a first outlet conduit in fluid communication with the first buffer pool and a second inlet conduit and a second outlet conduit in fluid communication with a second buffer pool for supplying and removing the buffer solution from, respectively, the first buffer pool and the second buffer pool.

10. The diagnostic system of claim 8, wherein the buffer solution flows at a rate of about 3 μl/min to about 8 μl/min through the first inlet conduit and the second inlet conduit to the first buffer pool and the second buffer pool and at a rate of about 1 μl/min to about 3 μl/min through the first buffer pool and the second buffer pool.

11. The diagnostic system of claim 1, the first electrode and the second electrode at least partially embedded in and extending, respectively, substantially orthogonal to a length of the electrophoresis strip in the first buffer pool and the second buffer pool.

12. A diagnostic system for identification and quantification of glycosylated hemoglobin and non-glycosylated hemoglobin in a blood sample, the diagnostic system comprising:
a) a cartridge that includes:
a housing including a microchannel extending between first and second buffer pools each containing a buffer solution, the buffer solution exhibiting an affinity to non-glycosylated hemoglobin, which facilitates its separation from glycosylated hemoglobin;
an electrophoresis strip positioned within the microchannel and structured to receive at least a portion of a hemolysate, the electrophoresis strip having first and second ends positioned in the first and second buffer pools so as to be at least partially saturated with the buffer solution in each buffer pool; and
a first electrode connected to the housing and exposed to the buffer solution in the first buffer pool and a second electrode connected to the housing and exposed to the buffer solution in the second buffer pool, the first and second electrodes configured to generate an electric field across the electrophoresis strip, the application of the electric field to the first and second electrodes inducing migration and separation of bands of non-glycosylated hemoglobin and glycosylated hemoglobin in the hemolysate delivered to the electrophoresis strip; wherein a portion of the housing is optically transparent for visualizing the migrated and separated bands of non-glycosylated hemoglobin and glycosylated hemoglobin on the electrophoresis strip;
b) a UV imaging system configured to illuminate the bands of non-glycosylated hemoglobin and glycosylated hemoglobin with UV radiation, detect through the optically transparent portion of the housing the bands of non-glycosylated hemoglobin and glycosylated hemoglobin on the electrophoresis strip caused by the applied electric field; and generate band detection data based on the bands of non-glycosylated hemoglobin and glycosylated hemoglobin; and
c) a processor configured to receive and analyze the band detection data to determine one or more band characteristics for the bands and generate diagnostic results based on the one or more band characteristics.

13. The diagnostic system of claim 12, wherein the UV imaging system is configured to detect at least one of band size, migration, and intensity and the processor includes a decision algorithm configured to generate density plots based on the band size, migration, and intensity and identify the relative percentages of glycosylated and non-glycosylated hemoglobin bands based on the generated density plots.

14. The diagnostic system of claim 13, wherein the processor is configured to diagnose whether the subject has or is at risk of diabetes.

15. The diagnostic system of claim 12, wherein the buffer solution is acidic and includes a sulfated polysaccharide.

16. The diagnostic system of claim 12, further comprising a power supply connected to the first electrode and the second electrode configured to supply the electric field across the electrophoresis strip.

17. The diagnostic system of claim 12, further comprising a buffer replenishing system configured to replenish the buffer solution for the first and second buffer pools and counteract pH changes in the buffer solution during operation of the system.

18. The diagnostic system of claim 17, wherein the buffer replenishing system includes a first inlet conduit and a first outlet conduit in fluid communication with the first buffer pool and a second inlet conduit and a second outlet conduit in fluid communication with a second buffer pool for supplying and removing the buffer solution from, respectively, the first buffer pool and the second buffer pool.

19. The diagnostic system of claim 17, wherein the buffer solution flows at a rate of about 3 μl/min to about 8 μl/min through the first inlet conduit and the second inlet to the first buffer pool and the second buffer pool and at a rate of about 1 μl/min to about 3 μl/min through the first buffer pool and the second buffer pool.
